SEQUENCE LISTING (107 sequences)

SEQ ID NO: 1 - protein, 33 aa, Homo sapiens
QPLPDCCRQKTCSCRLYELLHGAGNHAAGILTL

SEQ ID NO: 2 - protein, 33 aa, Gorilla; identical to SEQ ID NO: 1

SEQ ID NO: 3 - protein, 33 aa, Orangutan; identical to SEQ ID NO: 1

SEQ ID NO: 4 - protein, 33 aa, Chimpanzee; identical to SEQ ID NO: 1

SEQ ID NO: 5 - protein, 33 aa, Gibbon; identical to SEQ ID NO: 1

SEQ ID NO: 6 - protein, 33 aa, Green monkey; identical to SEQ ID NO: 1

SEQ ID NO: 7 - protein, 33 aa, Macaque; identical to SEQ ID NO: 1

SEQ ID NO: 8 - protein, 33 aa, Baboon; identical to SEQ ID NO: 1

SEQ ID NO: 9 - protein, 33 aa, Cow; identical to SEQ ID NO: 1

SEQ ID NO: 10 - protein, 33 aa, Rabbit; identical to SEQ ID NO: 1

SEQ ID NO: 11 - protein, 33 aa, Pig; identical to SEQ ID NO: 1

SEQ ID NO: 12 - protein, 33 aa, Dog; identical to SEQ ID NO: 1

SEQ ID NO: 13 - protein, 33 aa, Bat; identical to SEQ ID NO: 1

SEQ ID NO: 14 - protein, 33 aa, Yak; identical to SEQ ID NO: 1

SEQ ID NO: 15 - protein, 33 aa, Horse; identical to SEQ ID NO: 1

SEQ ID NO: 16 - protein, 33 aa, Squirrel; identical to SEQ ID NO: 1

SEQ ID NO: 17 - protein, 33 aa, Tupai; identical to SEQ ID NO: 1

SEQ ID NO: 18 - protein, 33 aa, Rat; identical to SEQ ID NO: 1

SEQ ID NO: 19 - protein, 33 aa, Mouse; identical to SEQ ID NO: 1

SEQ ID NO: 20 - protein, 33 aa, Otolemur; identical to SEQ ID NO: 1

SEQ ID NO: 21 - protein, 33 aa, Guinea Pig; identical to SEQ ID NO: 1

SEQ ID NO: 22 - protein, 33 aa, Polecat; identical to SEQ ID NO: 1

SEQ ID NO: 23 - protein, 33 aa, Marmoset
RLLPDCCRQKTCPCRRYELLHRGGNHAGGLLVL

SEQ ID NO: 24 - protein, 33 aa, Elephant; identical to SEQ ID NO: 1

SEQ ID NO: 25 - protein, 33 aa, Oppossum
QPMPNCCRQKTCSCRLYDLLHGAGNHAAGILTL

SEQ ID NO: 26 - protein, 33 aa, Tasmanian devil; identical to SEQ ID NO: 25

SEQ ID NO: 27 - protein, 33 aa, Chinese hamster; identical to SEQ ID NO: 1

SEQ ID NO: 28 - protein, 33 aa, Sheep; identical to SEQ ID NO: 1

SEQ ID NO: 29 - protein, 33 aa, Chicken
QSLPECCRQKTCSCRIYDLLHGMGNHAAGILTL

SEQ ID NO: 30 - protein, 33 aa, Alligator
QGVPDCCRQKTCSCRIYDLLHGMGNHAAGILTL

SEQ ID NO: 31 - protein, 33 aa, Penguin; identical to SEQ ID NO: 29

SEQ ID NO: 32 - protein, 33 aa, Amazon
QSLPECCRQKTCSCHIYDLLHGMGNHAAGILTL

SEQ ID NO: 33 - protein, 33 aa, Vulture; identical to SEQ ID NO: 29

SEQ ID NO: 34 - protein, 33 aa, Flycatcher
QNLPHCCRQKTCSCRVYDLLHGMGNHAAGILTL

SEQ ID NO: 35 - protein, 33 aa, Coelacanth
QSVPECCHQKTCSCRIYNLLHGNGNHAAGILTL

SEQ ID NO: 36 - protein, 33 aa, Hummingbird
QSLPECCRQKTCSCRVYDLLHGMGNHAAGILTL

SEQ ID NO: 37 - protein, 33 aa, Manakin
QSLPDCCRQKTCSCRVYDLLHGMGNHAAGILTL

SEQ ID NO: 38 - protein, 33 aa, zebra finch
HSLPHCCRQKTCPCRVYDLLHGMGNHAAGILTL

SEQ ID NO: 39 - protein, 33 aa, Grey
QNLPECCQQKTCSCRIYDLLHGMGNHAAGILTL

SEQ ID NO: 40 - protein, 33 aa, Chinese softshell
QSMPVCCRQKTCPCRVYDVLHGLGNHAAGILTL

SEQ ID NO: 41 - protein, 33 aa, Cobra
RAVPDCCRQKSCSCRIFDLLHGMGNHAAGILTI

SEQ ID NO: 42 - protein, 33 aa, Anole
QAVPDCCRQKSCPCNIFDLLHGTGNHANGILTL

SEQ ID NO: 43 - protein, 34 aa, Ray
SPRVPKCCCQQTCSCKVIDLLRGTGNHAAGILTL

SEQ ID NO: 44 - protein, 36 aa, Arowana
SQVRHCCRDGGDGTCPCRLYDVLRGRGDHAAGILTL

SEQ ID NO: 45 - protein, 33 aa, Xenopus
HGAPDCCRQKTCSCRIYDILRGTGNHAAGILTL

SEQ ID NO: 46 - protein, 49 aa, Zebra Fisch
EGVASCCARAPGSCKLYEMLCRAGRRNDSSVARHLVHLNNDAAVGILTL

SEQ ID NO: 47 - protein, 49 aa, Cod
SVSASCCSREPPRACRLYVLLLCGPVGGAGRALGGMHLGEDASAGILTL

SEQ ID NO: 48 - protein, 28 aa, Homo sapiens
RSGPPGLQGRLQRLLQASGNHAAGILTM

SEQ ID NO: 49 - protein, 28 aa, Gorilla
RSGPPGLQGRLQRLLQASGNHAAGILTM

SEQ ID NO: 50 - protein, 28 aa, Orangutan
RSGPPGLQGRLQRLLQASGNHAAGILTM

SEQ ID NO: 51 - protein, 28 aa, Chimpanzee
RSGPPGLQGRLQRLLQASGNHAAGILTM

SEQ ID NO: 52 - protein, 28 aa, Gibbon
RSGPPGLQGRLQRLLQASGNHAAGILTM

SEQ ID NO: 53 - protein, 28 aa, Green monkey
RSGPPGLQGRLQRLLQASGNHAAGILTM

SEQ ID NO: 54 - protein, 28 aa, Macaque
RSGPPGLQGRLQRLLQASGNHAAGILTM

SEQ ID NO: 55 - protein, 28 aa, Baboon
RSGPPGLQGRLQRLLQASGNHAAGILTM

SEQ ID NO: 56 - protein, 28 aa, Cow
RPGPPGLQGRLQRLLQASGNHAAGILTM

SEQ ID NO: 57 - protein, 28 aa, Rabbit
RPGPPGLQGRLQRLLQASGNHAAGILTM

SEQ ID NO: 58 - protein, 28 aa, Pig
RPGPPGLQGRLQRLLQASGNHAAGILTM

SEQ ID NO: 59 - protein, 28 aa, Dog
RPGPPGLQGRLQRLLQASGNHAAGILTM

SEQ ID NO: 60 - protein, 27 aa, Bat
RPGPGLQGRLQRLLQANGNHAAGILTM

SEQ ID NO: 61 - protein, 28 aa, Yak
RPGPPGLQGRLQRLLQASGNHAAGILTM

SEQ ID NO: 62 - protein, 28 aa, Horse
RPGPPGLHGRLQRLLQASGNHAAGILTM

SEQ ID NO: 63 - protein, 28 aa, Squirrel
RPGPPGLQGRLQRLLQASGNHAAGILTM

SEQ ID NO: 64 - protein, 28 aa, Tupai
RPGPPGLQGRLQRLLQASGNHAAGILTM

SEQ ID NO: 65 - protein, 28 aa, Rat
RPGPPGLQGRLQRLLQANGNHAAGILTM

SEQ ID NO: 66 - protein, 28 aa, Mouse
RPGPPGLQGRLQRLLQANGNHAAGILTM

SEQ ID NO: 67 - protein, 28 aa, Otolemur
RPGPPGLQGRLQRLLQASGNHAAGILTM

SEQ ID NO: 68 - protein, 28 aa, Guinea Pig
RPRPAGLQGRLQRLLLAGGNHAAGILTM

SEQ ID NO: 69 - protein, 28 aa, Polecat
RPGPPGLQGRLQRLLQASGNHAAGILTM

SEQ ID NO: 70 - protein, 28 aa, Marmoset
RAGPPNLQGQLQHLLQASGKHATGILTL

SEQ ID NO: 71 - protein, 28 aa, Elephant
RPGPPGLQGRLQRLLQASGNHAAGILTM

SEQ ID NO: 72 - protein, 28 aa, Oppossum
RAGLSGLQGRLQRLLQASGNHAAGILTV

SEQ ID NO: 73 - protein, 28 aa, Tasmanian devil
RAGLSGLQGRLQRLLQASGNHAAGILTV

SEQ ID NO: 74 - protein, 28 aa, Chinese hamster
RSGPPGLQGRLQRLLQASGNHAAGILTM

SEQ ID NO: 75 - protein, 28 aa, Sheep
RPGPPGLQGRLQRLLQASGNHAAGILTM

SEQ ID NO: 76 - protein, 28 aa, Chicken
KSIPPAFQSRLYRLLHGSGNHAAGILTM

SEQ ID NO: 77 - protein, 28 aa, Alligator
KSGSQPFQSRLYRLLQGSGNHAAGILTM

SEQ ID NO: 78 - protein, 28 aa, Penguin
KSVPPAFQSRLYRLLHSSGNHAAGILTM

SEQ ID NO: 79 - protein, 28 aa, Amazon
KSVPPAFQSRLYRLLHSSGNHAAGILTM

SEQ ID NO: 80 - protein, 28 aa, Vulture
KSIPPAFQSRLYRLLHGSGNHAAGILTM

SEQ ID NO: 81 - protein, 28 aa, Flycatcher
KSVPLAFQSRLYRLLHGSGNHAAGILTM

SEQ ID NO: 82 - protein, 29 aa, Coelacanth
KEAPPHAFQSRLYRLLHSPGNHAAGILTM

SEQ ID NO: 83 - protein, 28 aa, Hummingbird
KSIPSAFQSRLYHLLHSSGNHAAGILTM

SEQ ID NO: 84 - protein, 28 aa, Manakin
KSVPLAFQSRLYRLLHGSGNHAAGILTM

SEQ ID NO: 85 - protein, 28 aa, Zebra Finch
KSVPLTFQSRLYRLLHGSGNHAAGILTM

SEQ ID NO: 86 - protein, 28 aa, Grey
KSVLPAFQSRLYRLLHSSGNHAAGILTM

SEQ ID NO: 87 - protein, 28 aa, Chinese softshell
RSSSQAFQSQLYRLLHGSGNHAAGILTM

SEQ ID NO: 88 - protein, 30 aa, Cobra
SSAMTTKGFQSRLYRLLHGSENQAAGILTM

SEQ ID NO: 89 - protein, 30 aa, Anole
GSATATKTFQSRLYRLFHSSDNQAAGILTM

SEQ ID NO: 90 - protein, 28 aa, Ray
KTNAQPLQNRLHHLLHGLENQATGILTM

SEQ ID NO: 91 - protein, 28 aa, Arowana
QKPERHVQSRLYQLLQGSRDHTAGILTY

SEQ ID NO: 92 - protein, 28 aa, Xenopus
SDFQTMQSRLLQRLLQGSGNHAAGILTM

SEQ ID NO: 93 - protein, 28 aa, Zebra Fisch
KVGESRVHDRLQQLLHNSRNQAAGILTM

SEQ ID NO: 94 - protein, 29 aa, Cod
EAEEQHFHSRLHQLLRGGARNQAAGILTM

SEQ ID NO: 95 - protein, 11 aa, Homo sapiens
ASGNHAAGILT

SEQ ID NO: 96 - protein, 15 aa, Homo sapiens
ASGNHAAGILTMGRR

SEQ ID NO: 97 - protein, 22 aa, Homo sapiens
GLQGRLQRLLQASGNHAAGILT

SEQ ID NO: 98 - protein, 25 aa, Homo sapiens
GLQGRLQRLLQASGNHAAGILTGRR

SEQ ID NO: 99 - protein, 28 aa, Homo sapiens
RSGPPGLQGRLQRLLQASGNHAAGILTM

SEQ ID NO: 100 - protein, 29 aa, Homo sapiens
RSGPPGLQGRLQRLLQASGNHAAGILTMG

SEQ ID NO: 101 - protein, 30 aa, Homo sapiens
RSGPPGLQGRLQRLLQASGNHAAGILTMGR

SEQ ID NO: 102 - protein, 31 aa, Homo sapiens
RSGPPGLQGRLQRLLQASGNHAAGILTMGRR

SEQ ID NO: 103 - protein, 12 aa, Homo sapiens
GAGNHAAGILTL

SEQ ID NO: 104 - protein, 13 aa, Homo sapiens
GAGNHAAGILTLG

SEQ ID NO: 105 - protein, 449 aa, Artificial Sequence
  Synthetic VH cetuximab
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 214 aa, Artificial Sequence
  Synthetic VL cetuximab
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 12 aa, Artificial Sequence
  Synthetic fusion protein
NHAAGILTMGRR

(19) United States
(12) Patent Application Publication
(10) Pub. No.: US 2021/0188983 A1
(43) Pub. Date: Jun. 24, 2021

(54) POLYPEPTIDES FOR PREPARING DRUG CONJUGATES CAPABLE OF PROMOTING APOPTOSIS IN A CELL EXPRESSING AN OREXIN RECEPTOR

(71) Applicants: UNIVERSITE DE MONTPELLIER; INSTITUT REGIONAL DU CANCER DE MONTPELLIER; UNIVERSITE PARIS DIDEROT - PARIS 7

(72) Inventors: Bruno ROBERT, Montpelllier (FR); Pierre MARTINEAU, Montpellier Cedex 5 (FR); Alain COUVINEAU, Paris Cedex 18 (FR)

(21) Appl. No.: 16/077,946

(22) PCT Filed: Feb. 17, 2017
(86) PCT No.: PCT/EP2017/053616

§ 371 Date: Aug. 14, 2018

(30) Foreign Application Priority Data
Feb. 18, 2016 (EP) 16305187.3

(51) Int. Cl. C07K 16/28; A61K 47/68

(52) U.S. Cl. CPC C07K 16/2863; C07K 2319/30; A61K 47/6849; A61K 47/6803

(57) ABSTRACT

The present invention relates to polypeptides for preparing drug conjugates capable of promoting apoptosis in a cell expressing an orexin receptor. In particular, the present invention relates to a polypeptide comprising the amino acid sequence of formula of X20-X21-X22-X23-G-X25-L-X27-X28 wherein: —X20 represents N, D, or K, —X21 represents H, A or Q, —X22 represents A, S, T or G, —X23 represents A, T or G, —X25 represents I or L, —X27 represents T or V and, —X28 represents M, L, V, Y or I.

### FIELD OF THE INVENTION

The present invention relates to polypeptides for preparing drug conjugates capable of promoting apoptosis in a cell expressing an orexin receptor.

### BACKGROUND OF THE INVENTION

Orexins A and B (also known as hypocretins 1 and 2) are hypothalamic 33-aminoacid and 28-aminoacid neuropeptides, respectively, which originate from prepro-orexin, a 131-residue precursor. Orexin-A (OxA) contains two intramolecular disulfide bonds between positions 6 to 12 and 7 to 14 while orexin-B (OxB) does not have any. These two peptides share the same effects, regulating sleep, wakefulness, feeding, energy homeostasis, obesity, diabetes, breathing, reward system or drug addiction (Laburthe and Voisin, 2012). Orexins trigger biological effects by interacting with 2 members of the class A G-protein coupled receptor (GPCRs) family, i.e., orexin receptor-1 (OX1R) and orexin receptor-2 (OX2R) (Thompson et al., 2014). Activation of these receptors by orexins classically induces cellular calcium transients through Gq-dependent and -independent pathways (Laburthe et al., 2010). Besides these central actions, the orexins/receptor system is also involved in peripheral effects, including cardiovascular modulation, and neuroendocrine and reproduction regulation (Xu et al., 2013). Recently, our group demonstrated that OxA and OxB, bound to OX1R, can induce massive apoptosis, resulting in the drastic reduction of cell growth in various colonic cancer cell lines, including HT-29, LoVo, Caco-2 and others (Voisin et al., 2011). An entirely novel mechanism, not related to Gq-mediated phospholipase C activation, was shown to trigger orexin-induced apoptosis (Voisin et al., 2008; El Firar et al., 2009). In fact, orexins induced the tyrosine phosphorylation of two immunoreceptor tyrosine-based motifs (ITIMs) located at the interface between transmembrane domain (TM) 2 and TM 7 of OX1R and the cytoplasm (Voisin et al., 2008). The resulting phosphorylated receptor could then recruit and activate the phosphotyrosine phosphatase, SHP-2, which is responsible for mitochondrial apoptosis, involving cytochrome c release from mitochondria to cytosol and caspase-3 and caspase-7 activation (El Firar et al., 2009). The pro-apoptotic effect of orexins has also been extended to other cancer cell lines derived from human neuroblastoma (SK-N-MC cell line) and rat pancreatic cancer (AR42J cell line) (Rouet-Benzineb et al., 2004; Voisin et al., 2006). Recent data demonstrated that OX1R is aberrantly expressed in all resected primary colorectal tumors and liver metastases tested, but is not present in normal colon tissues (Voisin et al., 2011). Moreover, injection of exogenous orexins to mice strongly reduced in vivo tumor growth and reversed the development of established tumors in mice xenografted with colon cancer cell lines such as HT-29 or LoVo, due to robust apoptosis induction (Voisin et al., 2011). Taken together, these observations suggest that the orexins/OX1R system may represent a new promising target in colorectal cancer therapy, and most probably in other cancers, including pancreatic cancers neuroblastoma, and/or prostate cancer (Alexandre et al., 2014). In this context, structure-function relationship studies of the orexins/OX1R system are essential for the development of new agonists of OX1R that may represent new therapeutic approaches. The inventors recently explored the structure-function relationships of orexin-B (OxB) and OX1R (Br J Pharmacol. 2015 November; 172(21):5211-23). The contribution of all OxB residues in OxB-induced apoptosis was indeed investigated by alanine-scanning. Alanine substitution of OxB residues, L¹¹, L¹⁵, A²², G²⁴, I²⁵, L²⁶, and M²⁸, altered OxB binding affinity. Substitution of these residues and of the Q¹⁶, A¹⁷, S¹⁸, N²⁰ and T²⁷ residues inhibited apoptosis in CHO-S-OX1R cells. These results indicate that the C-terminus of OxB 1) plays an important role in the pro-apoptotic effect of the peptide; 2) interacts with some residues localized into the OX1R transmembrane domains.

### SUMMARY OF THE INVENTION

The present invention relates to polypeptides for preparing drug conjugates capable of promoting apoptosis in a cell expressing an orexin receptor. In particular, the present invention is defined by the claims.

### DETAILED DESCRIPTION OF THE INVENTION

The present invention relates to a polypeptide comprising the amino acid sequence of formula of X20-X21-X22-X23-G-X25-L-X27-X28 wherein:
X20 represents N, D, or K,
X21 represents H, A or Q,
X22 represents A, S, T or G,
X23 represents A, T or G,
X25 represents I or L,
X27 represents T or V and,
X28 represents M, L, V, Y or I.

As used herein the term “A” or “Ala” has its general meaning in the art and refers to Alanine. As used herein the term “R” or “Arg” has its general meaning in the art and refers to Arginine. As used herein the term “N” or “Asn” has its general meaning in the art and refers to Asparagine. As used herein the term “D” or “Asp” has its general meaning in the art and refers to Aspartic acid. As used herein the term “C” or “Cys” has its general meaning in the art and refers to Cysteine. As used herein the term “E” or “Glu” has its general meaning in the art and refers to Glutamic acid. As used herein the term “Q” or “Gln” has its general meaning in the art and refers to Glutamine. As used herein the term G or “Gly” has its general meaning in the art and refers to Glycine. As used herein the term “H” or “His” has its general meaning in the art and refers to Histidine. As used herein the term “I” or “Ile” has its general meaning in the art and refers to Isoleucine. As used herein the term “L” or “Leu” has its general meaning in the art and refers to Leucine. As used herein the term “K” or “Lys” has its general meaning in the art and refers to Lysine. As used herein the term “M” or “Met” has its general meaning in the art and refers to Methionine. As used herein the term “F” or “Phe” has its general meaning in the art and refers to Phenylalanine. As used herein the term “P” or “Pro” has its general meaning in the art and refers to Proline. As used herein the term “S” or “Ser” has its general meaning in the art and refers to Serine. As used herein the term “T” or “Thr” has its general meaning in the art and refers to Threonine. As used herein the term “W” or “Trp” has its general meaning in the art and refers to Tryptophan. As used herein the term “Y” or “Tyr” has its general meaning in the art and refers to Tyrosine. As used herein the term “V” or “Val” has its general meaning in the art and refers to Valine.

In some embodiments, the polypeptide of the present invention comprises or consists of 8; 9; 10; 11; 12; 13; 14; 15; 16; 17; 18; 19; 20; 21; 22; 23; 24; 25; 26; 27; 28; 29; 30; 31; 32; or 33 amino acids.

QPLPDCCRQKTCSCRLYELLHGAGNHAAGILTL (SEQ ID NO:1)

In some embodiments, the polypeptide of the present invention comprises an amino acid sequence having at least 50% of identity with the amino acid sequence ranging from the position 25 to the amino acid at position 33 in SEQ ID NO:1.

In some embodiments, the polypeptide of the present invention comprises an amino acid sequence having at least 50% of identity with the amino acid sequence ranging from the position 24 to the amino acid at position 33 in SEQ ID NO:1.

In some embodiments, the polypeptide of the present invention comprises an amino acid sequence having at least 50% of identity with the amino acid sequence ranging from the position 23 to the amino acid at position 33 in SEQ ID NO:1.

In some embodiments, the polypeptide of the present invention comprises an amino acid sequence having at least 50% of identity with the amino acid sequence ranging from the position 22 to the amino acid at position 33 in SEQ ID NO:1.

In some embodiments, the polypeptide of the present invention comprises an amino acid sequence having at least 50% of identity with the amino acid sequence ranging from the position 21 to the amino acid at position 33 in SEQ ID NO:1.

In some embodiments, the polypeptide of the present invention comprises an amino acid sequence having at least 50% of identity with the amino acid sequence ranging from the position 20 to the amino acid at position 33 in SEQ ID NO:1.

In some embodiments, the polypeptide of the present invention comprises an amino acid sequence having at least 50% of identity with the amino acid sequence ranging from the position 19 to the amino acid at position 33 in SEQ ID NO:1.

In some embodiments, the polypeptide of the present invention comprises an amino acid sequence having at least 50% of identity with the amino acid sequence ranging from the position 18 to the amino acid at position 33 in SEQ ID NO:1.

In some embodiments, the polypeptide of the present invention comprises an amino acid sequence having at least 50% of identity with the amino acid sequence ranging from the position 17 to the amino acid at position 33 in SEQ ID NO:1.

In some embodiments, the polypeptide of the present invention comprises an amino acid sequence having at least 50% of identity with the amino acid sequence ranging from the position 16 to the amino acid at position 33 in SEQ ID NO:1.

In some embodiments, the polypeptide of the present invention comprises an amino acid sequence having at least 50% of identity with the amino acid sequence ranging from the position 15 to the amino acid at position 33 in SEQ ID NO:1.

In some embodiments, the polypeptide of the present invention comprises an amino acid sequence selected from the group of SEQ ID NO:1-47 as described in FIG. 1.

(SEQ ID NO: 48)
RSGPPGLQGRLQRLLQASGNHAAGILTM
(20-28)

In some embodiments, the polypeptide of the present invention comprises an amino acid sequence having at least 50% of identity with the amino acid sequence ranging from the position 20 to the amino acid at position 28 in SEQ ID NO:48.

In some embodiments, the polypeptide of the present invention comprises an amino acid sequence having at least 50% of identity with the amino acid sequence ranging from the position 19 to the amino acid at position 28 in SEQ ID NO:48.

In some embodiments, the polypeptide of the present invention comprises an amino acid sequence having at least 50% of identity with the amino acid sequence ranging from the position 18 to the amino acid at position 28 in SEQ ID NO:48.

In some embodiments, the polypeptide of the present invention comprises an amino acid sequence having at least 50% of identity with the amino acid sequence ranging from the position 17 to the amino acid at position 28 in SEQ ID NO:48.

In some embodiments, the polypeptide of the present invention comprises an amino acid sequence having at least 50% of identity with the amino acid sequence ranging from the position 16 to the amino acid at position 28 in SEQ ID NO:48.

In some embodiments, the polypeptide of the present invention comprises an amino acid sequence having at least 50% of identity with the amino acid sequence ranging from the position 15 to the amino acid at position 28 in SEQ ID NO:48.

In some embodiments, the polypeptide of the present invention comprises an amino acid sequence having at least 50% of identity with the amino acid sequence ranging from the position 14 to the amino acid at position 28 in SEQ ID NO:48.

In some embodiments, the polypeptide of the present invention comprises an amino acid sequence having at least 50% of identity with the amino acid sequence ranging from the position 13 to the amino acid at position 28 in SEQ ID NO:48.

In some embodiments, the polypeptide of the present invention comprises an amino acid sequence having at least 50% of identity with the amino acid sequence ranging from the position 12 to the amino acid at position 28 in SEQ ID NO:48.

In some embodiments, the polypeptide of the present invention comprises an amino acid sequence having at least 50% of identity with the amino acid sequence ranging from the position 11 to the amino acid at position 28 in SEQ ID NO:48.

In some embodiments, the polypeptide of the present invention comprises an amino acid sequence having at least 50% of identity with the amino acid sequence ranging from the position 10 to the amino acid at position 28 in SEQ ID NO:48.

In some embodiments, the polypeptide of the present invention comprises an amino acid sequence selected from the group of SEQ ID NO:48-94 as described in FIG. 2.

According to the present invention a first amino acid sequence having at least 50% of identity with a second amino acid sequence means that the first sequence has 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; or 100% of identity with the second amino acid sequence.

Sequence identity is frequently measured in terms of percentage identity (or similarity or homology); the higher the percentage, the more similar are the two sequences. Methods of alignment of sequences for comparison are well known in the art. Various programs and alignment algorithms are described in: Smith and Waterman, Adv. Appl. Math., 2:482, 1981; Needleman and Wunsch, J. Mol. Biol., 48:443, 1970; Pearson and Lipman, Proc. Natl. Acad. Sci. U.S.A., 85:2444, 1988; Higgins and Sharp, Gene, 73:237-244, 1988; Higgins and Sharp, CABIOS, 5:151-153, 1989; Corpet et al. Nuc. Acids Res., 16:10881-10890, 1988; Huang et al., Comp. Appls Biosci., 8:155-165, 1992; and Pearson et al., Meth. Mol. Biol., 24:307-31, 1994). Altschul et al., Nat. Genet., 6:119-129, 1994, presents a detailed consideration of sequence alignment methods and homology calculations. By way of example, the alignment tools ALIGN (Myers and Miller, CABIOS 4:11-17, 1989) or LFASTA (Pearson and Lipman, 1988) may be used to perform sequence comparisons (Internet Program® 1996, W. R. Pearson and the University of Virginia, fasta20u63 version 2.0u63, release date December 1996). ALIGN compares entire sequences against one another, while LFASTA compares regions of local similarity. These alignment tools and their respective tutorials are available on the Internet at the NCSA Website, for instance. Alternatively, for comparisons of amino acid sequences of greater than about 30 amino acids, the Blast 2 sequences function can be employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). The BLAST sequence comparison system is available, for instance, from the NCBI web site; see also Altschul et al., J. Mol. Biol., 215:403-410, 1990; Gish. & States, Nature Genet., 3:266-272, 1993; Madden et al. Meth. Enzymol., 266:131-141, 1996; Altschul et al., Nucleic Acids Res., 25:3389-3402, 1997; and Zhang & Madden, Genome Res., 7:649-656, 1997.

In some embodiments, the polypeptide of the present invention is extended at its c-terminal end by at least one amino acid. In some embodiments, the polypeptide of the present invention is extended at its c-terminal end by at least one glycine (G). In some embodiments, the polypeptide of the present invention is extended at its c-terminal end by at least 2 amino acids. In some embodiments, the polypeptide of the present invention is extended at its c-terminal end by the amino acid sequence GR or GK. In some embodiments, the polypeptide of the present invention is extended at its c-terminal end by at least 3 amino acids. In some embodiments, the polypeptide of the present invention is extended at its c-terminal end by the amino acid sequence GRR, GRK, GKR, or GKK.

In some embodiments, the polypeptide of the present invention comprises an amino acid sequence selected from the group consisting of SEQ ID NO:95-104.

(SEQ ID NO: 95)
ASGNHAAGILT
(SEQ ID NO: 96)
ASGNHAAGILTMGRR
(SEQ ID NO: 97)
GLQGRLQRLLQASGNHAAGILT
(SEQ ID NO: 98)
GLQGRLQRLLQASGNHAAGILTGRR
(SEQ ID NO: 99)
RSGPPGLQGRLQRLLQASGNHAAGILTM
(SEQ ID NO: 100)
RSGPPGLQGRLQRLLQASGNHAAGILTMG
(SEQ ID NO: 101)
RSGPPGLQGRLQRLLQASGNHAAGILTMGR
(SEQ ID NO: 102)
RSGPPGLQGRLQRLLQASGNHAAGILTMGRR
(SEQ ID NO: 103)
GAGNHAAGILTL
(SEQ ID NO: 104)
GAGNHAAGILTLG

The polypeptide of the present is capable of binding to an orexin receptor. In some embodiments, the polypeptide of the present invention is capable of binding to the OX1R. In some embodiments, the polypeptide of the present invention is capable of promoting the apoptosis of a cell expressing an orexin receptor. In some embodiments, the polypeptide of the present invention is capable of promoting the apoptosis of a cell expressing the OX1R receptor. As used herein, the tem “OX1R” has its general meaning in the art and refers to the 7-transmembrane spanning receptor OX1R for orexins. According to the invention, OX1R promotes apoptosis in cancer cells through a mechanism which is not related to Gq-mediated phopholipase C activation and cellular calcium transients. The polypeptide of the present invention can induce tyrosine phosphorylation of 2 tyrosine-based motifs in OX1R, ITIM and ITSM, resulting in the recruitment of the phosphotyrosine phosphatase SHP-2, the activation of which is responsible for mitochondrial apoptosis (Voisin T, El Firar A, Rouyer-Fessard C, Gratio V, Laburthe M. A hallmark of immunoreceptor, the tyrosine-based inhibitory motif ITIM, is present in the G protein-coupled receptor OX1R for orexins and drives apoptosis: a novel mechanism. FASEB J. 2008 June; 22(6):1993-2002; El Firar A, Voisin T, Rouyer-Fessard C, Ostuni M A, Couvineau A, Laburthe M. Discovery of a functional immunoreceptor tyrosine-based switch motif in a 7-transmembrane-spanning receptor: role in the orexin receptor OX1R-driven apoptosis. FASEB J. 2009 December; 23(12):4069-80. doi: 10.1096/fj.09-131367. Epub Aug. 6, 2009). The capability of the polypeptide of the present invention to promote apoptosis can be assessed by any assay well known in the art. Typically, the apoptosis assay typically involve use of CHO-S cells expressing recombinant native or mutated OX1R that are seeded and grown. After 24 hr culture, cells are treated with or without the polypeptide to be tested. After 48 hr of treatment, adherent cells were harvested. Apoptosis is then determined using the Guava PCA system and the Guava nexin kit. Results are expressed as the percentage of apoptotic annexin V-phycoerythrin (PE)-positive cells. According to the invention, the polypeptide of the present invention keeps the same activity than Orexin-B. Typically, the apoptosis induction (EC50) of the polypeptide of the present invention ranges from 10 nM to 110 nM. More particularly, the apoptosis induction (EC50) of the polypeptide of the present invention ranges from 10 nM to 50 nM. More particularly, the apoptosis induction (EC50) of the polypeptide of the present invention ranges from 15 nM to 30 nM.

The polypeptides of the present invention may be produced by any suitable means, as will be apparent to those of skill in the art. In order to produce sufficient amounts of polypeptides or functional equivalents thereof for use in accordance with the present invention, expression may conveniently be achieved by culturing under appropriate conditions recombinant host cells containing the polypeptide of the present invention. In particular, the polypeptide is produced by recombinant means, by expression from an encoding nucleic acid molecule. Systems for cloning and expression of a polypeptide in a variety of different host cells are well known. When expressed in recombinant form, the polypeptide is in particular generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells, HeLa cells, baby hamster kidney cells and many others. Bacteria are also preferred hosts for the production of recombinant protein, due to the ease with which bacteria may be manipulated and grown. A common, preferred bacterial host is E. coli. Alternatively, the polypeptide of the present invention is produced by any technique known in the art, such as, without limitation, any chemical, biological, genetic or enzymatic technique, either alone or in combination. For example, knowing the amino acid sequence of the desired sequence, one skilled in the art can readily produce said polypeptide, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions.

A further aspect of the present invention relates to a nucleic acid encoding for a polypeptide of the present invention. As used herein, the term “nucleic acid molecule” has its general meaning in the art and refers to a DNA or RNA molecule. However, the term captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxylmethyl) uracil, 5-fiuorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine. In some embodiments, the nucleic acid molecule of the present invention is included in a suitable vector, such as a plasmid, cosmid, episome, artificial chromosome, phage or a viral vector. So, a further object of the invention relates to a vector comprising a nucleic acid encoding for a polypeptide of the invention. Typically, the vector is a viral vector which is an adeno-associated virus (AAV), a retrovirus, bovine papilloma virus, an adenovirus vector, a lentiviral vector, a vaccinia virus, a polyoma virus, or an infective virus. In some embodiments, the vector is an AAV vector.

A further object of the present invention relates to a host cell transformed with the nucleic acid molecule of the present invention. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”. For instance, as disclosed above, for expressing and producing the polypeptide of the present invention, prokaryotic cells and, in particular E. coli cells, will be chosen. Actually, according to the invention, it is not mandatory to produce the polypeptides of the present invention in a eukaryotic context that will favour post-translational modifications (e.g. glycosylation). Typically, the host cell may be suitable for producing the polypeptide of the present invention as described above. In some embodiments, the host cells is isolated from a mammalian subject who is selected from a group consisting of: a human, a horse, a dog, a cat, a mouse, a rat, a cow and a sheep. In some embodiments, the host cell is a human cell. In some embodiments, the host cell is a cell in culture. The cells may be obtained directly from a mammal (preferably human), or from a commercial source, or from tissue, or in the form for instance of cultured cells, prepared on site or purchased from a commercial cell source and the like. In some embodiments, the host cell is a mammalian cell line (e.g., Vero cells, CHO cells, 3T3 cells, COS cells, etc.).

A further object of the present invention relates to a drug conjugate wherein the polypeptide of the present invention is linked to a targeting moiety. In some embodiments, the polypeptide of the present invention is linked with its N-terminal end to a targeting moiety. In some embodiments, the polypeptide of the present invention is linked with its C-terminal end to a targeting moiety.

As used herein, the term “targeting moiety” refers to any molecule that binds specifically to a target. In some embodiments, the targeting moiety is selected from the group consisting of aptamers and polypeptides (e.g. ligands).

In some embodiments, the targeting moiety is capable of binding to a cell expressing the orexin receptor. In some embodiments, the targeting moiety has binding affinity to a cell surface molecule of a cell expressing an orexin receptor. In some embodiments, the cell surface molecule is a receptor. In some embodiments, the cell surface molecule is a transmembrane protein. In some embodiments, the targeting moiety targets a tumor-associated antigen. As used herein, “tumor-associated antigens” means any antigen which is generally associated with tumor cells, i.e., occurring at the same or to a greater extent as compared with normal cells. Such antigens may be relatively tumor specific and limited in their expression to the surface of malignant cells, although they may also be found on non-malignant cells. Exemplary tumor-associated antigens bound by the starting polypeptides used in the invention include for example, pan B antigens (e.g. CD20 found on the surface of both malignant and non-malignant B cells such as those in non-Hodgkin's lymphoma) and pan T cell antigens (e.g. CD2, CD3, CD5, CD6, CD7). Other exemplary tumor associated antigens comprise but are not limited to MAGE-1, MAGE-3, MUC-1, HPV 16, HPV E6 & E7, TAG-72, CEA, α-Lewisy, L6-Antigen, CD19, CD22, CD25, CD30, CD33, CD37, CD44, CD52, CD56, mesothelin, PSMA, HLA-DR, EGF Receptor, VEGF Receptor, and HER2 Receptor. Carcinoembryonic antigen (CEA), and α-fetoprotein (AFP) are two examples of such tumor associated antigens. Other targets include the MICA/B ligands of NKG2D. These molecules are expressed on many types of tumors, but not normally on healthy cells. Additional specific examples of tumor associated antigens include epithelial cell adhesion molecule (Ep-CAM/TACSTD1), mesothelin, tumor-associated glycoprotein 72 (TAG-72), gp100, Melan-A, MART-1, KDR, RCAS1, MDA7, cancer-associated viral vaccines (e.g., human papillomavirus antigens), prostate specific antigen (PSA, PSMA), RAGE (renal antigen), CAMEL (CTL-recognized antigen on melanoma), CT antigens (such as MAGE-B5, -B6, -C2, -C3, and D; Mage-12; CT10; NY-ESO-1, SSX-2, GAGE, BAGE, MAGE, and SAGE), mucin antigens (e.g., MUC1, mucin-CA125, etc.), cancer-associated ganglioside antigens, tyrosinase, gp75, C-myc, Mart1, MelanA, MUM-1, MUM-2, MUM-3, HLA-B7, Ep-CAM, tumor-derived heat shock proteins, and the like (see also, e.g., Acres et al., Curr Opin Mol Ther 2004 February, 6:40-7; Taylor-Papadimitriou et al., Biochim Biophys Acta. 1999 Oct. 8; 1455(2-3):301-13; Emens et al., Cancer Biol Ther. 2003 July-August; 2(4 Suppl 1):S161-8; and Ohshima et al., Int J Cancer. 2001 Jul. 1; 93(1):91-6). Other exemplary tumor associated antigen targets include CA 195 tumor-associated antigen-like antigen (see, e.g., U.S. Pat. No. 5,324,822) and female urine squamous cell carcinoma-like antigens (see, e.g., U.S. Pat. No. 5,306,811), and the breast cell tumor associated antigens described in U.S. Pat. No. 4,960,716.

As used herein the term “aptamer” has its general meaning in the art and refers to nucleic or amino acid targeting macromolecules that may be designed to bind tightly to specific target molecules. Peptide aptamers are short peptides of random amino acid sequences. As commonly used, these peptides are generally 15-20 amino acids-long. This length provides enough flexibility for the peptide to assume various conformations, while reducing the probability of randomly creating a stop codon in the aptamer coding sequence. In some embodiments, the apatmer is any polynucleotide, generally a RNA or a DNA that has a useful biological activity in terms of biochemical activity, molecular recognition or binding attributes.

In some embodiments, the targeting moiety is a heterologous polypeptide (i.e., a polypeptide other than the same polypeptide of the present invention) having a binding domain. The term “binding domain” as used herein refers to the one or more regions of a polypeptide that mediate specific binding with a target molecule (e.g. an antigen, ligand, receptor, substrate or inhibitor). Exemplary binding domains include an antibody variable domain, a receptor binding domain of a ligand, a ligand binding domain of a receptor or an enzymatic domain. The term “ligand binding domain” as used herein refers to any native receptor (e.g., cell surface receptor) or any region or derivative thereof retaining at least a qualitative ligand binding ability of a corresponding native receptor. The term “receptor binding domain” as used herein refers to any native ligand or any region or derivative thereof retaining at least a qualitative receptor binding ability of a corresponding native ligand. In some embodiments, the heterologous polypeptide comprises at least 1, 2, 3, 4, or 5 binding sites. The polypeptide may be either monomers or multimers. For example, in some embodiments, the heterologous polypeptide is a dimer. In some embodiments, the dimer is an homodimer, comprising two identical monomeric subunits. In some embodiments, the dimer is an heterodimer, comprising two non-identical monomeric subunits. The subunits of the dimer may comprise one or more polypeptide chains. For example, in some embodiments, the dimer comprises at least two polypeptide chains. In some embodiments, the dimer comprises two polypeptide chains. In some embodiments, the dimer comprises four polypeptide chains (e.g., as in the case of antibody molecules). In some embodiments, the targeting moiety is an antibody. The term “antibody” is thus used to refer to any antibody-like molecule that has an antigen binding region, and this term includes antibody fragments that comprise an antigen binding domain such as Fab′, Fab, F(ab′)2, single domain antibodies (DABs or VHH), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art. In some embodiments, the antibody is a monoclonal antibody. In some embodiments, the antibody is non-internalizing. As used herein the term “non-internalizing antibody” refer to an antibody, respectively, that has the property of to bind to a target antigen present on a cell surface, and that, when bound to its target antigen, does not enter the cell and become degraded in the lysosome. In some embodiments, the heterologous polypeptide is a light immunoglobulin chain. In some embodiments, the heterologous polypeptide is a heavy immunoglobulin chain. In some embodiments, the heterologous polypeptide is a heavy single heavy chain variable domain of antibodies of the type that can be found in Camelid mammals which are naturally devoid of light chains. Such single domain antibody are also called VHH or “Nanobody®”. For a general description of (single) domain antibodies, reference is also made to the prior art cited above, as well as to EP 0 368 684, Ward et al. (Nature Oct. 12, 1989; 341 (6242): 544-6), Holt et al., Trends Biotechnol., 2003, 21(11):484-490; and WO 06/030220, WO 06/003388.

In some embodiments, targeting moiety is a monoclonal antibody selected from the group consisting of Abciximab, Adalimumab, Ado-trastuzumab emtansine, Alemtuzumab, Basiliximab, Belimumab, Bevacizumab, Blinatumomab, Brentuximab vedotin, Canakinumab, Catumaxomab, Certolizumab pegol, Cetuximab, Daclizumab, Denosumab, Dinutuximab, Eculizumab, Efalizumab, Evolocumab, Gemtuzumab ozogamicin, Golimumab, Ibritumomab tiuxetan, Infliximab, Ipilimumab, Mepolizumab, Muromonab-CD3, Natalizumab, Necitumumab, Nivolumab, Obinutuzumab, Ofatumumab, Omalizumab, Palivizumab, Panitumumab, Pembrolizumab, Pertuzumab, Ramucirumab, Ranibizumab, Raxibacumab, Rituximab, Secukinumab, Siltuximab, Tocilizumab, Tositumomab, Trastuzumab, Ustekinumab and Vedolizumab.

In some embodiments, the targeting moiety is cetuximab. As used herein the term “cetuximab” has its general meaning in the art and refers to the antibody characterized by the heavy chain as set forth in SEQ ID NO:105 and the light chain as set forth in SEQ ID NO:106.

SEQ ID NO: 105: Cetuximab H γ1
QVQLKQSGPGLVQPSQSLSITCTVSGFSLTNYGVHWVRQSPGKGLEW
LGVIWSGGNTDYNTPFTSRLSINKDNSKSQVFFKMNSLQSNDTAIYY
CARALTYYDYEFAYWGQGTLVTVSAASTKGPSVFPLAPSSKSTSGGT
AALGCLVKDYFPEPVTVSWNSGALTSGVHTFPAVLQSSGLYSLSSVV
TVPSSSLGTQTYICNVNHKPSNTKVDKRVEPKSCDKTHTCPPCPAPE
LLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKA
LPAPIEKTISKAKGQPREPQVYTLPPSREEMTKNQVSLTCLVKGFYP
SDIAVEWESNGQPENNYKTTPPVLDSDGSFFLYSKLTVDKSRWQQGN
VFSCSVMHEALHNHYTQKSLSLSPGK
SEQ ID NO: 106: Cetuximab L kappa
DILLTQSPVILSVSPGERVSFSCRASQSIGTNIHWYQQRTNGSPRLL
IKYASESISGIPSRFSGSGSGTDFTLSINSVESEDIADYYCQQNNNW
PTTFGAGTKLELKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC

In some embodiments, the polypeptide of the present invention is conjugated to the targeting moiety. As used herein, the term “conjugation” has its general meaning in the art and means a chemical conjugation. Techniques for conjugating targeting moiety to polypeptides, are well-known in the art (See, e.g., Arnon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58. See also, e.g., PCT publication WO 89/12624.) Typically, the nucleic acid molecule is covalently attached to lysines or cysteines on the antibody, through N-hydroxysuccinimide ester or maleimide functionality respectively. Methods of conjugation using engineered cysteines or incorporation of unnatural amino acids have been reported to improve the homogeneity of the conjugate (Axup, J. Y., Bajjuri, K. M., Ritland, M., Hutchins, B. M., Kim, C. H., Kazane, S. A., Halder, R., Forsyth, J. S., Santidrian, A. F., Stafin, K., et al. (2012). Synthesis of site-specific antibody-drug conjugates using unnatural amino acids. Proc. Natl. Acad. Sci. USA 109, 16101-16106; Junutula, J. R., Flagella, K. M., Graham, R. A., Parsons, K. L., Ha, E., Raab, H., Bhakta, S., Nguyen, T., Dugger, D. L., Li, G., et al. (2010). Engineered thio-trastuzumab-DM1 conjugate with an improved therapeutic index to target human epidermal growth factor receptor 2-positive breast cancer. Clin. Cancer Res.16, 4769-4778). Junutula et al. (2008) developed cysteine-based site-specific conjugation called “THIOMABs” (TDCs) that are claimed to display an improved therapeutic index as compared to conventional conjugation methods. In particular the one skilled in the art can also envisage a polypeptide engineered with an acyl donor glutamine-containing tag (e.g., Gin-containing peptide tags or Q-tags) or an endogenous glutamine that are made reactive by polypeptide engineering (e.g., via amino acid deletion, insertion, substitution, or mutation on the polypeptide). Then a transglutaminase, can covalently crosslink with an amine donor agent (e.g., a small molecule comprising or attached to a reactive amine) to form a stable and homogenous population of an engineered Fc-containing polypeptide conjugate with the amine donor agent being site-specifically conjugated to the Fc-containing polypeptide through the acyl donor glutamine-containing tag or the accessible/exposed/reactive endogenous glutamine (WO 2012059882). The term “transglutaminase”, used interchangeably with “TGase” or “TG”, refers to an enzyme capable of cross-linking proteins through an acyl-transfer reaction between the γ-carboxamide group of peptide-bound glutamine and the ε-amino group of a lysine or a structurally related primary amine such as amino pentyl group, e.g. a peptide-bound lysine, resulting in a ε-(γ-glutamyl)lysine isopeptide bond. TGases include, inter alia, bacterial transglutaminase (BTG) such as the enzyme having EC reference EC 2.3.2.13 (protein-glutamine-γ-glutamyltransferase). In some embodiments, the polypeptide of the present invention is conjugated to the targeting moiety by a linker molecule. As used herein, the term “linker molecule” refers to any molecule attached to the polypeptide of the present invention. The attachment is typically covalent. In some embodiments, the linker molecule is flexible and does not interfere with the binding of the polypeptide of the present invention.

In some embodiments, when the targeting moiety is a heterologous polypeptide, the polypeptide of the present invention is fused to the heterologous polypeptide to form a fusion protein. As used herein, a “fusion protein” comprises the polypeptide of the present invention operably linked to a heterologous polypeptide. Within the fusion protein, the term “operably linked” is intended to indicate that the polypeptide of the present invention and the heterologous polypeptide are fused in-frame to each other. The heterologous polypeptide can be fused to the N-terminus or C-terminus of the polypeptide of the present invention. In some embodiment, the heterologous polypeptide is fused to the C-terminal end of the polypeptide of the present invention. In some embodiments, the polypeptide of the present invention and the heterologous polypeptide are fused to each other directly (i.e. without use of a linker) or via a linker. The linker is typically a linker peptide and will, according to the invention, be selected so as to allow binding of the polypeptide to the heterologous polypeptide. Suitable linkers will be clear to the skilled person based on the disclosure herein, optionally after some limited degree of routine experimentation. Suitable linkers are described herein and may—for example and without limitation—comprise an amino acid sequence, which amino acid sequence preferably has a length of 2 or more amino acids. Typically, the linker has 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. However, the upper limit is not critical but is chosen for reasons of convenience regarding e.g. biopharmaceutical production of such fusion proteins. The linker sequence may be a naturally occurring sequence or a non-naturally occurring sequence. If used for therapeutical purposes, the linker is preferably non-immunogenic in the subject to which the fusion protein of the present invention is administered. One useful group of linker sequences are linkers derived from the hinge region of heavy chain antibodies as described in WO 96/34103 and WO 94/04678. Other examples are poly-alanine linker sequences such as Ala-Ala-Ala. Further preferred examples of linker sequences are Gly/Ser linkers of different length including (gly4ser)3, (gly4ser)4, (gly4ser), (gly3ser), gly3, and (gly3ser2)3.

In some embodiments, the present invention relates to a fusion protein wherein the polypeptide of the present invention which is extended by the GRR amino acid is fused by its c-terminal end to a heterologous polypeptide. In some embodiments, the present invention relates to a fusion protein comprising the amino acid sequence NHAAGILTMGRR fused by its c-terminal end to a heterologous polypeptide.

In some embodiments, the drug conjugate of the present invention is both capable of targeting a cell by binding to the surface molecule and promoting apoptosis of the cell by binding to the orexin receptor expressed by the cell. In some embodiments, the cell is a cancer cell. Accordingly, the drug conjugate of the present invention (including the fusion protein described above) is suitable for the treatment of cancer.

Accordingly a further object of the present invention relates to a method of treating cancer in a subject in need thereof comprising administering to the subject a therapeutically effective amount of the drug conjugate of the present invention.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of cancer. The methods of the present invention contemplate any one or more of these aspects of treatment.

Typically, the cancer may be selected from the group consisting of bile duct cancer (e.g. periphilar cancer, distal bile duct cancer, intrahepatic bile duct cancer), bladder cancer, bone cancer (e.g. osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma, lymphoma, multiple myeloma), brain and central nervous system cancer (e.g. meningioma, astocytoma, oligodendrogliomas, ependymoma, gliomas, medulloblastoma, ganglioglioma, Schwannoma, germinoma, craniopharyngioma), breast cancer (e.g. ductal carcinoma in situ, infiltrating ductal carcinoma, infiltrating, lobular carcinoma, lobular carcinoma in, situ, gynecomastia), Castleman disease (e.g. giant lymph node hyperplasia, angiofollicular lymph node hyperplasia), cervical cancer, colorectal cancer, endometrial cancer (e.g. endometrial adenocarcinoma, adenocanthoma, papillary serous adnocarcinroma, clear cell), esophagus cancer, gallbladder cancer (mucinous adenocarcinoma, small cell carcinoma), gastrointestinal carcinoid tumors (e.g. choriocarcinoma, chorioadenoma destruens), Hodgkin's disease, non-Hodgkin's lymphoma, Kaposi's sarcoma, kidney cancer (e.g. renal cell cancer), laryngeal and hypopharyngeal cancer, liver cancer (e.g. hemangioma, hepatic adenoma, focal nodular hyperplasia, hepatocellular carcinoma), lung cancer (e.g. small cell lung cancer, non-small cell lung cancer), mesothelioma, plasmacytoma, nasal cavity and paranasal sinus cancer (e.g. esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyosarcoma), salivary gland cancer, skin cancer (e.g. melanoma, nonmelanoma skin cancer), stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma, thyroid lymphoma), vaginal cancer, vulvar cancer, and uterine cancer (e.g. uterine leiomyosarcoma).

In some embodiments, the subject suffers from an epithelial cancer. As used herein, the term “epithelial cancer” refers to any malignant process that has an epithelial origin. Examples of epithelial cancers include, but are not limited to, a gynecological cancer such as endometrial cancer, ovarian cancer, cervical cancer, vulvar cancer, uterine cancer or fallopian tube cancer, breast cancer, prostate cancer, lung cancer, pancreatic cancer, urinary cancer, bladder cancer, head and neck cancer, oral cancer colorectal cancer and liver cancer. An epithelial cancer may be at different stages as well as varying degrees of grading. In some embodiments, the epithelial cancer is selected from the group consisting of breast cancer, prostate cancer, lung cancer, pancreatic cancer, bladder cancer colorectal cancer and ovarian cancer. In some embodiments, the epithelial cancer is a colorectal cancer. In some embodiments, the epithelial cancer is a liver cancer, in particular a hepatocellular carcinoma. In some embodiments, the epithelial cancer is breast cancer. In some embodiments, the epithelial cancer is ovarian cancer. In some embodiments, the epithelial cancer is prostate cancer, in particular advanced prostate cancer. In some embodiments, the epithelial cancer is lung cancer. In some embodiments, the epithelial cancer is head and neck cancer. In some embodiments, the epithelial cancer is head and neck squamous cell carcinoma.

As used herein the term “pancreatic cancer” or “pancreas cancer” as used herein relates to cancer which is derived from pancreatic cells. In particular, pancreatic cancer included pancreatic adenocarcinoma (e.g., pancreatic ductal adenocarcinoma) as well as other tumors of the exocrine pancreas (e.g., serous cystadenomas), acinar cell cancers, intraductal papillary mucinous neoplasms (IPMN) and pancreatic neuroendocrine tumors (such as insulinomas).

As used herein the term “hepatocellular carcinoma” has its general meaning in the art and refers to the cancer developed in hepatocytes. In general, liver cancer indicates hepatocellular carcinoma in large. HCC may be caused by an infectious agent such as hepatitis B virus (HBV, hereinafter may be referred to as HBV) or hepatitis C virus (HCV, hereinafter may be referred to as HCV). In some embodiments, HCC results from alcoholic steatohepatitis or non-alcoholic steatohepatitis (hereinafter may be abbreviated to as “NASH”). In some embodiments, the HCC is early stage HCC, non-metastatic HCC, primary HCC, advanced HCC, locally advanced HCC, metastatic HCC, HCC in remission, or recurrent HCC. In some embodiments, the HCC is localized resectable (i.e., tumors that are confined to a portion of the liver that allows for complete surgical removal), localized unresectable (i.e., the localized tumors may be unresectable because crucial blood vessel structures are involved or because the liver is impaired), or unresectable (i.e., the tumors involve all lobes of the liver and/or has spread to involve other organs (e.g., lung, lymph nodes, bone). In some embodiments, the HCC is, according to TNM classifications, a stage I tumor (single tumor without vascular invasion), a stage II tumor (single tumor with vascular invasion, or multiple tumors, none greater than 5 cm), a stage III tumor (multiple tumors, any greater than 5 cm, or tumors involving major branch of portal or hepatic veins), a stage IV tumor (tumors with direct invasion of adjacent organs other than the gallbladder, or perforation of visceral peritoneum), N1 tumor (regional lymph node metastasis), or M1 tumor (distant metastasis). In some embodiments, the HCC is, according to AJCC (American Joint Commission on Cancer) staging criteria, stage T1, T2, T3, or T4 HCC.

As used herein the term “advanced prostate cancer” has its general meaning in the art. “Castration resistant prostate cancer,” “CaP,” “androgen-receptor dependent prostate cancer,” “androgen-independent prostate cancer,” are used interchangeably to refer to prostate cancer in which prostate cancer cells “grow” {i.e., increase in number) in the absence of androgens and/or in the absence of expression of androgen receptors on the cancer cells.

In some embodiments, the drug conjugate of the present invention (in particular, when the targeting moiety is cetuximab) is particularly suitable for the treatment of metastatic colorectal cancer, in particular metastatic colorectal cancer associated with at least one RAS mutation, in particular at least one KRAS mutation. The term “RAS mutation” has its general meaning in the art and refers to the mutations in the Ras family of proto-oncogenes (comprising H-Ras, N-Ras, K-Ras, DIRAS1; DIRAS2; DIRAS3; ERAS; GEM; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1: REM2; RERG; RERGL; RRAD; RRAS; RRAS2). In particular, the term “KRAS mutation” includes any one or more mutations in the KRAS (which can also be referred to as KRAS2 or RASK2) gene. For example, the KRAS mutations are located in exon 3 or exon 4 of the gene. Examples of KRAS mutations include, but are not limited to, G12C, G12D, G13D, G12R, G12S, and G12V. KRAS is one of the commonly mutated oncogenes in human cancers. In particular, KRAS mutations are found in 30-40% of tumors and represent together with APC one of the somatic alteration involved in the initiation of colorectal cancer. This mutation occurs early in the process of carcinogenesis, and is maintained at the various stages of disease progression, such as node involvement and metastatic spread. A recent study involving a large number of patients has demonstrated that mutated KRAS is associated with worse outcome in colorectal cancer progression, with effects being more pronounced in stage II and III disease (Nash, et al., Ann. Surg. Oncol., 17: 416-424, 2010). The same group has shown, in another study (Nash, et al., Ann. Surg. Oncol., 17: 572-578, 2010), that KRAS mutation is associated with more rapid and aggressive metastatic behavior of colorectal liver metastases. In addition, KRAS mutation has been reported to induce drug resistance and treatment failure to epidermal-growth factor receptor (EGFR)-targeting therapeutics in metastatic colorectal cancer. KRAS mutations confer resistance to both cetuximab (Erbitux®) and panitumumab (Vectibix®) (Allegra et al., J. Clin. Oncol., 27: 2091-2096, 2008; Linardou et al., Lancet Oncol., 9: 962-972, 2008).

In some embodiments, the drug conjugate of the present invention (in particular, when the targeting moiety is cetuximab) is particularly suitable for the treatment of metastatic colorectal cancer, in particular metastatic colorectal cancer associated with at least one BRAF mutation. The term “BRAF mutation” includes any one or more mutations in the BRAF (which can also be referred to as serine/threonine-protein kinase B-Raf or B-Raf) gene. Typically, the BRAF mutation is V600E. The serine-threonine kinase BRAF is the principal effector of KRAS and BRAF wild-type had been shown to be required for response to panitumumab or cetuximab and is used to select patients who are eligible for the treatment.

As used herein, the term “therapeutically effective amount” refers to an amount effective, at dosages and for periods of time necessary, to achieve a desired therapeutic result. A therapeutically effective amount of a drug conjugate of the present invention may vary according to factors such as the disease state, age, sex, and weight of the individual, and the ability of the conjugate of the present invention to elicit a desired response in the individual. The efficient dosages and dosage regimens for the drug conjugate of the present invention linked to the targeting moiety depend on the disease or condition to be treated and may be determined by the persons skilled in the art. A physician having ordinary skill in the art may readily determine and prescribe the effective amount of the pharmaceutical composition required. For example, the physician could start doses of drug conjugate employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. The ability of a compound to inhibit cancer may, for example, be evaluated in an animal model system predictive of efficacy in human tumors. Alternatively, this property of a composition may be evaluated by examining the ability of the compound to inhibit cell growth or to induce cytotoxicity by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected. An exemplary, non-limiting range for a therapeutically effective amount of a drug conjugate of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of a polypeptide of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. In some embodiments, the efficacy may be monitored by measuring the level of OX1R in a sample containing tumor cells, by visualization of the disease area, or by other diagnostic methods described further herein, e.g. by performing one or more PET-CT scans, for example using a labeled polypeptide of the present invention, fragment or mini-antibody derived from drug conjugate. If desired, an effective daily dose of a pharmaceutical composition may be administered as two, three, four, five, six or more sub-doses administered separately at appropriate intervals throughout the day, optionally, in unit dosage forms. In some embodiments, the drug conjugates of the present invention are administered by slow continuous infusion over a long period, such as more than 24 hours, in order to minimize any unwanted side effects. An effective dose of a drug conjugate of the present invention may also be administered using a weekly, biweekly or triweekly dosing period. The dosing period may be restricted to, e.g., 8 weeks, 12 weeks or until clinical progression has been established. As non-limiting examples, treatment according to the present invention may be provided as a daily dosage of a compound of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

For administration, drug conjugate is formulated as a pharmaceutical composition. A pharmaceutical composition comprising a drug conjugate of the present invention can be formulated according to known methods to prepare pharmaceutically useful compositions, whereby the therapeutic molecule is combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. (See, e.g., Gennaro (ed.), Remington's Pharmaceutical Sciences (Mack Publishing Company, 19th ed. 1995)) Formulations may further include one or more excipients, preservatives, solubilizers, buffering agents, albumin to prevent protein loss on vial surfaces, etc. The form of the pharmaceutical compositions, the route of administration, the dosage and the regimen naturally depend upon the condition to be treated, the severity of the illness, the age, weight, and sex of the patient, etc. The pharmaceutical compositions of the present invention can be formulated for a topical, oral, parenteral, intranasal, intravenous, intramuscular, subcutaneous or intraocular administration and the like. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The doses used for the administration can be adapted as a function of various parameters, and in particular as a function of the mode of administration used, of the relevant pathology, or alternatively of the desired duration of treatment. To prepare pharmaceutical compositions, an effective amount of drug conjugate may be dissolved or dispersed in a pharmaceutically acceptable carrier or aqueous medium. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. A drug conjugate can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. The drug conjugates of the present invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered. In addition to the compounds formulated for parenteral administration, such as intravenous or intramuscular injection, other pharmaceutically acceptable forms include, e.g. tablets or other solids for oral administration; time release capsules; and any other form currently used. In some embodiments, the use of liposomes and/or nanoparticles is contemplated for the introduction of antibodies into host cells. The formation and use of liposomes and/or nanoparticles are known to those of skill in the art. Nanocapsules can generally entrap compounds in a stable and reproducible way. To avoid side effects due to intracellular polymeric overloading, such ultrafine particles (sized around 0.1 μm) are generally designed using polymers able to be degraded in vivo. Biodegradable polyalkyl-cyanoacrylate nanoparticles that meet these requirements are contemplated for use in the present invention, and such particles may be are easily made. Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core. The physical characteristics of liposomes depend on pH, ionic strength and the presence of divalent cations.

The invention will be further illustrated by the following figures and examples. However, these examples and figures should not be interpreted in any way as limiting the scope of the present invention.

### FIGURES

FIG. 1 show different orexin-A polypeptides.

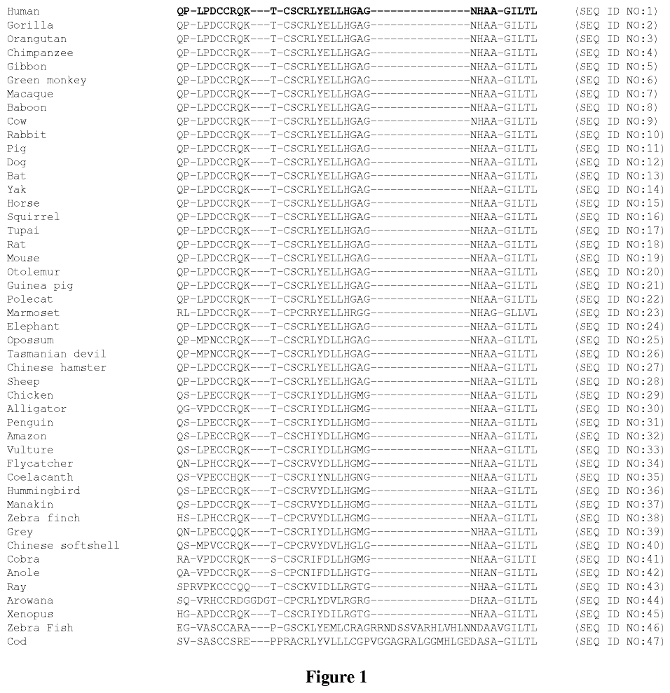

FIG. 2 show different orexin-B polypeptides.

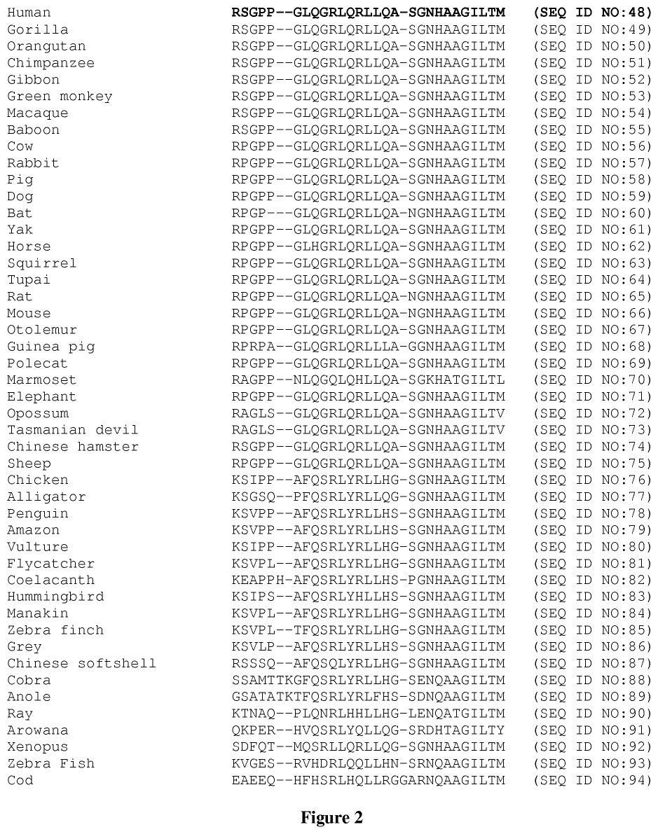

FIG. 3: Effect of orexin-B (OxB) and various constructions (referenced in Table 1) on cell growth of HEK-OX1R cells. 0.1 μM of OxB or 0.1 μM of compounds were incubated with HEK-OX1R cells for 48 h. After incubation cells were counted and results were expressed as the percentage of the number of untreated cells (control). NS, non significant; *, p<0.05; **, p<0.01; ***, p<0.001.

FIG. 4: Effect of orexin-B (OxB) and various constructions (referenced in Table 1) on cell growth of HEK-OX1R cells. 0.1 μM of OxB or 0.1 μM of compounds were incubated with HEK-OX1R cells for 48 h. After incubation cells were counted and results were expressed as the percentage of the number of untreated cells (control). NS, non significant; *, p<0.05; **, p<0.01; ***, p<0.001.

FIG. 5: Effect of compounds on cell viability of HEK-OX1R, HT-29, LoVo and AsPC-1 cells determined by using the WST-1 kit (Roche). A-B. Various concentration of OxA or OxB or 20 μg/ml C11 or 20 μg/ml C12 were incubated with HEK-OX1R, HT-29 (colon cancer), LoVo (colon cancer) and AsPC-1 (pancreas cancer) cells for 48 h. Cell viability was determined using WST-1 kit accordingly to the manufacturer's instructions, p<0.01; ***, p<0.001.

### EXAMPLE

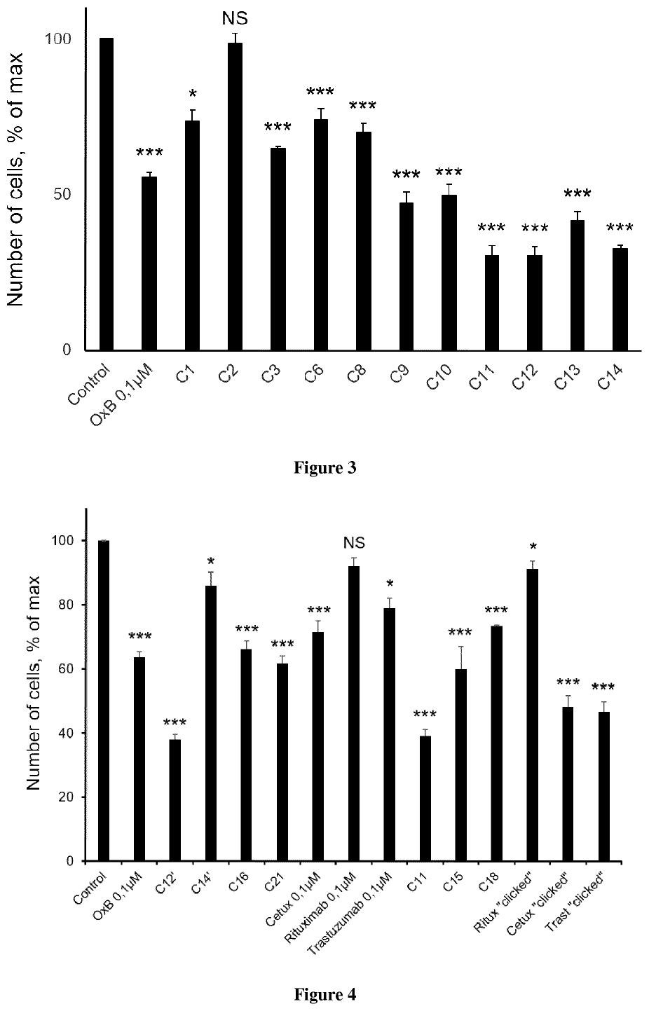

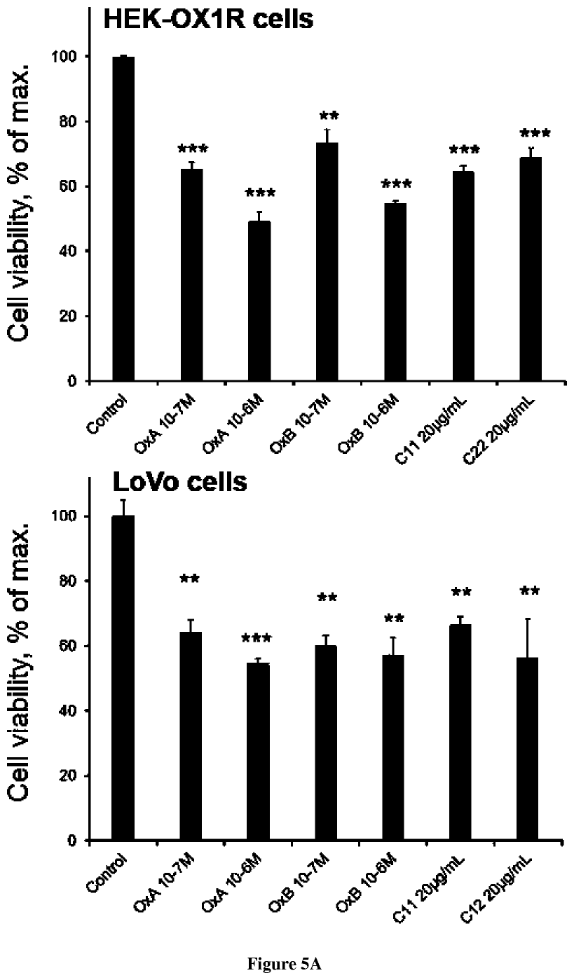

The inventors prepared different constructions comprising different orexin polypeptides were assayed for their inhibition on cell growth. In particular, the orexin polypeptides were linked (fused or conjugated) to different antibodies (i.e. cetuximab, rituximab, and trastuzumab) on their light (LC) and/or heavy chains (HC). The antibodies were full antibodies, F(ab)2 or F(ab). The conjugations (“click”) were realized following the teaching of Transglutaminase-Based Chemo-Enzymatic Conjugation Approach Yields Homogeneous Antibody-Drug Conjugates. Dennler, P. et al., Bioconjugate Chemistry 2013, and Site-Specific Conjugation of Monomethyl Auristatin E to Anti-CD30 Antibodies Improves Their Pharmacokinetics and Therapeutic Index in Rodent Models. Lhospice, F. et al. Mol. Pharm. (2015). All the constructions are described in Table 1 with their effect on the inhibition of cell growth. FIGS. 3 and 4 show the different constructions on cell growth of HEK-OX1R cells.

TABLE 1
Constructions and their effects
on the inhibition of cell growth.

| | | Inhibition |
|---|---|---|
| | | of cell |
| Name | Construction | growth, % |
| OxB | RSGPPGLQGRLQRLLQASGNHAAGILTM | 40 |
| OxA | QPLPDCCRQKTCSCRLYELLHGAGNHAAGILTL | 52 |
| C8 | Cetux mAb-(LC)- | 30 |
| | RSGPPGLQGRLQRLLQASGNHAAGILTM | |
| C9 | Cetux mAb-(LC)- | 51 |
| | RSGPPGLQGRLQRLLQASGNHAAGILTMGRR | |
| C1 | Cetux mAb-(HC)- | 30 |
| | RSGPPGLQGRLQRLLQASGNHAAGILTMGRR | |
| C2 | Cetux mAb-(LC + HC)- | 37 |
| | RSGPPGLQGRLQRLLQASGNHAAGILTMGRR | |
| C3 | Cetux F(ab)2-(LC)- | 35 |
| | RSGPPGLQGRLQRLLQASGNHAAGILTMGRR | |
| C6 | Cetux F(ab)-(LC)- | 26 |
| | RSGPPGLQGRLQRLLQASGNHAAGILTMGRR | |
| C10 | Cetux mAb-(LC)- | 50 |
| | RSGPPGLQGRLQRLLQASGNHAAGILTMGR | |
| C11 | Cetux mAb-(LC)- | 65 |
| | RSGPPGLQGRLQRLLQASGNHAAGILTMG | |
| C12 | Cetux mAb-(LC)- | 65 |
| | GLQGRLQRLLQASGNHAAGILTMGRR | |
| C12′ | Cetux mAb-(LC)- | 68 |
| | GLQGRLQRLLQASGNHAAGILTMG | |
| C13 | Cetux mAb-(LC)- | 58 |
| | ASGNHAAGILTMGRR | |
| C14 | RSGPPGLQGRLQRLLQASGNHAAGILTMGRR- | 65 |
| | Cetux mAb-(LC) | |
| C14′ | RSGPPGLQGRLQRLLQASGNHAAGILTMG- | 14 |
| | Cetux mAb-(LC) | |
| C15 | Trastuz mAb-(LC)- | 40 |
| | RSGPPGLQGRLQRLLQASGNHAAGILTMG | |
| C16 | Cetux mAb-(LC)-NHAAGILTMG | 34 |
| C18 | Ritux mAb-(LC)- | 30 |
| | RSGPPGLQGRLQRLLQASGNHAAGILTMG | |
| C21 | Cetux mAb-(LC)-GAGNHAAGILTLG(OxA) | 38 |
| Ritux | Ritux mAb-click- | 10 |
| « clicked » | RSGPPGLQGRLQRLLQASGNHAAGILTM-NH2 | |
| Cetux | Cetux mAb-click- | 52 |
| « clicked » | RSGPPGLQGRLQRLLQASGNHAAGILTM-NH2 | |
| Trast | Trastuz mAb-click- | 54 |
| « clicked » | RSGPPGLQGRLQRLLQASGNHAAGILTM-NH2 | |
| Rituximab | Ritux mAb | NS |
| Trastuzumab | Trastuz mAb | 20 |
| Cetuximab | Cetux mAb | 30 |

Results are expressed as the percentage of inhibition of HEK-OX1R cell growth, assuming that untreated cells displays no inhibition (0%).
NS, non-significatif.
mAb, full IgG1;
HC, heavy chain;
LC, light chain;
Ritux, Rituximab;
Cetux, Cetuximab;
Trastuz, Trastuzumab.
Click, conjugaison on Q295 of peptide using Transglutaminase.

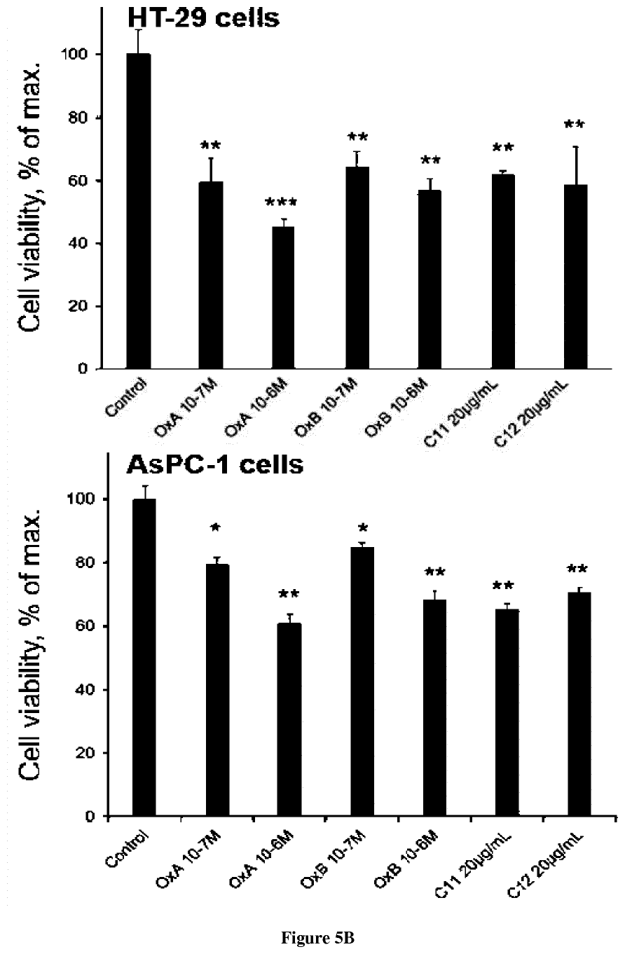

As shown in FIG. 5, OxA and OxB reduce the cell viability of recombinant HEK-OX1R cells and also cancer cell lines derived from colonic and pancreatic adenocarcinoma. These effects were dose-dependent (FIGS. 5 A and B). Similarly, C11 and C12 induced the inhibition of cells viability of these cell lines as compared to orexins impact (FIGS. 5 A and B).

### REFERENCES

Throughout this application, various references describe the state of the art to which this invention pertains. The disclosures of these references are hereby incorporated by reference into the present disclosure.

1. A polypeptide comprising the amino acid sequence of formula of X20-X21-X22-X23TG-X25-L-X27-X28 wherein:
X20 represents N, D, or K,
X21 represents H, A or Q
X22 represents A, S, T or G,
X23 represents A, T or G,
X25 represents I or L,
X27 represents T or V and,
X28 represents M, L, V, Y or 1.

2. The polypeptide of claim 1 which comprises or consists of 8; 9; 10; 11; 12; 13; 14; 15; 16; 17; 18; 19; 20; 21; 22; 23; 24; 25; 26; 27; 28; 29; 30; 31; 32; or 33 amino acids.

3. The polypeptide of claim 1 which comprises:
an amino acid sequence having at least 50% of identity with the amino acid sequence ranging from the position 25 to the amino acid at position 33 in SEQ ID NO:1
an amino acid sequence having at least 50% of identity with the amino acid sequence ranging from the position 24 to the amino acid at position 33 in SEQ ID NO:1
an amino acid sequence having at least 50% of identity with the amino acid sequence ranging from the position 23 to the amino acid at position 33 in SEQ ID NO:1
an amino acid sequence having at least 50% of identity with the amino acid sequence ranging from the position 22 to the amino acid at position 33 in SEQ ID NO:1
an amino acid sequence having at least 50% of identity with the amino acid sequence ranging from the position 21 to the amino acid at position 33 in SEQ ID NO:1
an amino acid sequence having at least 50% of identity with the amino acid sequence ranging from the position 20 to the amino acid at position 33 in SEQ ID NO:1
an amino acid sequence having at least 50% of identity with the amino acid sequence ranging from the position 19 to the amino acid at position 33 in SEQ ID NO:1
an amino acid sequence having at least 50% of identity with the amino acid sequence ranging from the position 18 to the amino acid at position 33 in SEQ ID NO:1
an amino acid sequence having at least 50% of identity with the amino acid sequence ranging from the position 17 to the amino acid at position 33 in SEQ ID NO:1
an amino acid sequence having at least 50% of identity with the amino acid sequence ranging from the position 16 to the amino acid at position 33 in SEQ ID NO:1 or
an amino acid sequence having at least 50% of identity with the amino acid sequence ranging from the position 15 to the amino acid at position 33 in SEQ ID NO:1

4. The polypeptide of claim 1 which comprises an amino acid sequence selected from the group consisting of SEQ ID N NOS:1-47.

5. The polypeptide of claim 1 which comprises:
an amino acid sequence having at least 50% of identity with the amino acid sequence ranging from the position 20 to the amino acid at position 28 in SEQ ID NO:48
an amino acid sequence having at least 50% of identity with the amino acid sequence ranging from the position 19 to the amino acid at position 28 in SEQ ID NO:48
an amino acid sequence having at least 50% of identity with the amino acid sequence ranging from the position 18 to the amino acid at position 28 in SEQ ID NO:48
an amino acid sequence having at least 50% of identity with the amino acid sequence ranging from the position 17 to the amino acid at position 28 in SEQ ID NO:48
an amino acid sequence having at least 50% of identity with the amino acid sequence ranging from the position 16 to the amino acid at position 28 in SEQ ID NO:48
an amino acid sequence having at least 50% of identity with the amino acid sequence ranging from the position 15 to the amino acid at position 28 in SEQ ID NO:48
an amino acid sequence having at least 50% of identity with the amino acid sequence ranging from the position 14 to the amino acid at position 28 in SEQ ID NO:48
an amino acid sequence having at least 50% of identity with the amino acid sequence ranging from the position 13 to the amino acid at position 28 in SEQ ID NO:48
an amino acid sequence having at least 50% of identity with the amino acid sequence ranging from the position 12 to the amino acid at position 28 in SEQ ID NO:48
an amino acid sequence having at least 50% of identity with the amino acid sequence ranging from the position 11 to the amino acid at position 28 in SEQ ID NO:48 or
an amino acid sequence having at least 50% of identity with the amino acid sequence ranging from the position 10 to the amino acid at position 28 in SEQ ID NO:48.

6. The polypeptide of claim 1 which comprises an amino acid sequence selected from the group consisting of SEQ ID NOS:48-94.

7. The polypeptide of claim 1 which is extended at its c-terminal end by at least one amino acid.

8. The polypeptide of claim 1 which is extended at its c-terminal end by at least 2 amino acids.

9. The polypeptide of claim 1 which is extended at its c-terminal end by at least 3 amino acids.

10. The polypeptide of claim 1 which comprises an amino acid sequence selected from the group consisting of SEQ ID NOS:95-104.

11. A nucleic acid encoding the polypeptide of claim 1.

12. A drug conjugate comprising the polypeptide of claim 1 linked to a targeting moiety.

13. The drug conjugate of claim 12 wherein the polypeptide is linked with its N-terminal end to the targeting moiety, or the polypeptide is linked with its C-terminal end to the targeting moiety.

14. The drug conjugate of claim 12 wherein the targeting moiety is selected from the group consisting of aptamers and polypeptides.

15. The drug conjugate of claim 12 wherein the targeting moiety has binding affinity to a cell surface molecule of a cell expressing an orexin receptor.

16. The drug conjugate of claim 12 wherein the targeting moiety targets a tumor-associated antigen.

17. The drug conjugate of claim 12 wherein the targeting moiety is an antibody.

18. The drug conjugate of claim 12 wherein the targeting moiety is a monoclonal antibody selected from the group consisting of Abiximab, Adalimumab, Ado-trastuzumab emtansine, Alemtuzumab, Basiliximab, Belimumab, Bevacizurnab, Blinatumomab, Brentuximab vedotin, Canakinumab, Catumaxomab, Certolizumab pegol, Cetuximab, Daclizunab, Denosumab, Dinutuximab, Eculizumab, Efalizumab, Evolocumab, Gertuzumab ozogamicin, Golimumab, lbriturmomab tiuxetan, Infliximab, Ipilimumab, Mepolizumab, Muromonab-CD3, Natalizumab, Necitumumab, Nivolumab, Obinutuzunab, Ofatumumab, Omalizumab, Palivizumab, Panitumumab, Pembrolizurnab, Pertuzumab, Ramucirumab, Ranibizumab, Raxibacumab, Rituximab, Secukinumab, Siltuximab, Tocilizumab, Tositumomab, Trastuzumab, Ustekinumab and Vedolizumab.

19. The drug conjugate of claim 12 wherein the targeting moiety is cetuximab.

20. A fusion protein wherein the polypeptide of claim 1 that is extended by the amino acids GRR and is fused by its c-terminal end to a heterologous polypeptide.

21. A method of treating cancer in a subject in need thereof comprising administering to the subject a therapeutically effective amount of the drug conjugate of claim 12.

22. A pharmaceutical composition comprising the drug conjugate of claim 12.

23. The polypeptide of claim 7 wherein the at least one amino acid is glycine.

24. The polypeptide of claim 8 wherein the at least 2 amino acids are GR or GK.

25. The polypeptide of claim 9 wherein the at least 3 amino acids are GRR, GRK, GKR, or GKK.
